(19) United States
(12) Patent Application Publication
(10) Pub. No.: US 2016/0368844 A1
(43) Pub. Date: Dec. 22, 2016

(54) Process for Producing Mixtures of Cyclohexanone and Cyclohexanol

(71) Applicant: EXXONMOBIL CHEMICAL PATENTS INC., Baytown, TX (US)

(72) Inventors: Jihad M. Dakka, Whitehouse Station, NJ (US); Charles M. Smith, Princeton, NJ (US); Kun Wang, Bridgewater, NJ (US); Francisco M. Benitez, Cypress, TX (US); Roberto Garcia, Easton, PA (US); John W. Chu, Neshanic Station, NJ (US); Keith H. Kuechler, Friendswood, TX (US); Chuansheng Bai, Phillipsburg, NJ (US)

(21) Appl. No.: 14/901,601

(22) PCT Filed: Mar. 7, 2014
(86) PCT No.: PCT/US2014/045421

§ 371 Date: Dec. 28, 2015

Related U.S. Application Data
(60) Provisional application No. 61/857,813, filed on Jul. 24, 2013.

(30) Foreign Application Priority Data
Oct. 2, 2013 (EP) 13187106.3

(51) Int. Cl. C07C 29/20; B01J 21/08; B01J 23/14; B01J 21/04; B01J 23/42; B01J 23/44

(52) U.S. Cl. CPC C07C 29/20; B01J 23/42; B01J 23/44; B01J 23/14; B01J 21/04; B01J 21/08

(57) ABSTRACT

In a process for producing a mixture of cyclohexanone and cyclohexanol, a feed comprising cyclohexanone is contacted with hydrogen in the presence of a hydrogenation catalyst under hydrogenation conditions effective to convert part of the cyclohexanone in the feed into cyclohexanol and thereby produce a hydrogenation product containing cyclohexanone and cyclohexanol. A mixture of cyclohexanone and cyclohexanol is then obtained from the hydrogenation product.

### PRIORITY CLAIM TO RELATED APPLICATIONS

This application claims priority to U.S. Provisional Application Ser. No. 61/857,813 filed Jul. 24, 2013 and European Application No. 13187106.3 filed Oct. 2, 2013, the disclosures of which are fully incorporated herein by their reference.

### FIELD

The present invention relates to a process for producing mixtures of cyclohexanone and cyclohexanol.

### BACKGROUND

Mixtures of cyclohexanone and cyclohexanol, also known as KA oil, are used as precursors for the production of Nylon-6,6. KA oil is currently produced by the catalytic oxidation of cyclohexane. However, the conversion is very low (on the order of 3-5%) and the selectivity to the desired ketone/alcohol mixture is only 70-80%. There is therefore interest in finding alternative processes for the production of mixtures of cyclohexanone and cyclohexanol.

It is known that benzene can be converted to cyclohexylbenzene via in-situ hydroalkylation over bifunctional catalysts which comprise hydrogenation and acid functionalities. The resultant cyclohexylbenzene can then be oxidized to cyclohexylbenzene hydroperoxide, which in turn can be cleaved to make an approximately 50/50 molar ratio of phenol and cyclohexanone. While this process provides an attractive alternative to the existing Hock process for the production of phenol, one potential challenge facing the technology is that cyclohexanone and phenol produce an azeotropic mixture comprising about 28 wt % cyclohexanone and about 72 wt % phenol. Thus, it is difficult to obtain all of the cyclohexanone, or any of the phenol, from the cleavage effluent as a saleable product with reasonable purity by simple traditional distillation. An extractive solvent can be added into the mixture to break the azeotrope during distillation. However, the extractive solvent has to be removed in a subsequent step, adding to the complexity and cost of the process. Therefore, alternative solutions dealing with the handling of the cleavage effluent are desired.

As one solution, U.S. Published Patent Application No. 2011/0105805 discloses a process for producing phenol by oxidation of cyclohexylbenzene to cyclohexylbenzene hydroperoxide followed by cleavage of the cyclohexylbenzene hydroperoxide, in which some or all of the effluent from the cleavage step is subjected to a selective dehydrogenation step to convert at least part of the cyclohexanone in the effluent portion into phenol and hydrogen.

WO2013/052216 discloses another solution in which the production of phenol from the effluent of cyclohexylbenzene hydroperoxide cleavage is enhanced by separating cyclohexanone from the effluent, typically by conventional distillation, to leave an effluent fraction with reduced cyclohexanone content typically approaching the azeotropic amount of 28 wt %. This effluent fraction is then subjected to dehydrogenation to convert at least part of the cyclohexanone into phenol.

### SUMMARY

According to the invention, a further solution to the problem of handling the mixture of phenol and cyclohexanone resulting from the cleavage of cyclohexylbenzene hydroperoxide has now been found in which at least a portion of the cleavage effluent is hydrogenated to produce a mixture of cyclohexanol and cyclohexanone (i.e., KA oil) from the resulting hydrogenation product. In this way, the present process reduces the need for complex and costly separation steps while at the same time provides flexibility to meet the market demand for three important chemical products, namely phenol, cyclohexanone and KA oil.

More particularly, in one aspect, the invention relates to a process for producing a mixture of cyclohexanone and cyclohexanol, the process comprising:

(a) contacting a feed comprising cyclohexanone and phenol with hydrogen in the presence of a hydrogenation catalyst under hydrogenation conditions effective to convert at least part of the cyclohexanone and/or phenol in the feed into cyclohexanol and thereby produce a hydrogenation product containing cyclohexanone and cyclohexanol.

In embodiments of this aspect, the process further comprises:

(b) obtaining a mixture comprising cyclohexanone and cyclohexanol from the hydrogenation product.

In a further aspect, the invention relates to a process for producing a mixture of cyclohexanone and cyclohexanol, the process comprising:

(a) cleaving a cleavage feed comprising cyclohexylbenzene hydroperoxide to produce a cleavage effluent comprising phenol and cyclohexanone;

(b) obtaining a hydrogenation feed comprising phenol and/or cyclohexanone from at least a portion of the cleavage effluent;

(c) contacting the hydrogenation feed with hydrogen in the presence of a hydrogenation catalyst in a hydrogenation reaction zone under hydrogenation conditions effective to convert at least part of the phenol and/or cyclohexanone in the hydrogenation feed into cyclohexanol and thereby produce a hydrogenation product containing cyclohexanone and cyclohexanol.

In embodiments of this aspect, the process further comprises:

(d) obtaining a mixture comprising cyclohexanone and cyclohexanol from the hydrogenation product.

In yet a further aspect, the invention relates to a hydrogenation catalyst comprising platinum, palladium, tin and an inorganic oxide material.

### BRIEF DESCRIPTION OF THE DRAWINGS

FIG. 1 is a flow diagram of a process for producing phenol, cyclohexanone and KA oil from benzene according to one example of the invention.

FIG. 2 is a graph showing concentrations of cyclohexanol, cyclohexanone and phenol against time on stream for the hydrogenation process of Example 10.

FIG. 3 is a graph showing concentrations of light and heavy by-products against time on stream for the hydrogenation process of Example 10.

FIG. 4 is a graph showing concentrations of cyclohexanol, cyclohexanone and phenol against time on stream for the hydrogenation process of Example 11.

FIG. 5 is a graph showing concentrations of light and heavy by-products against time on stream for the hydrogenation process of Example 11.

FIG. 6 is a bar chart comparing concentrations of cyclohexanol, cyclohexanone and phenol and light and heavy by-products for the catalysts of Examples 2 and 3 in the hydrogenation process of Example 12.

FIG. 7 is a bar chart comparing concentrations of cyclohexanol, cyclohexanone and phenol and light and heavy by-products for the catalysts of Examples 2, 4 and 5 in the hydrogenation process of Example 13.

FIG. 8 is a bar chart comparing concentrations of cyclohexanol, cyclohexanone and phenol and light and heavy by-products for the catalysts of Examples 1, 2, 7, 8, 9, and 10 in the hydrogenation process of Example 14.

### DETAILED DESCRIPTION OF THE EMBODIMENTS

In the present disclosure, a process may be described as comprising at least one “step.” It should be understood that each step is an action or operation that may be carried out once or multiple times in the process, in a continuous or discontinuous fashion. Unless specified to the contrary or the context clearly indicates otherwise, each step in a process may be conducted sequentially in the order as they are listed, with or without overlapping with one or more other step, or in any other order, as the case may be. In addition, some steps may be conducted simultaneously, for example, in the same reaction zone.

Unless otherwise indicated, all numbers in the present disclosure are to be understood as being modified by the term “about” in all instances. It should also be understood that the precise numerical values used in the specification and claims constitute specific embodiments. Efforts have been made to ensure the accuracy of the data in the examples. However, it should be understood that any measured data inherently contain a certain level of error due to the limitation of the technique and equipment used for making the measurement.

As used herein, the indefinite articles “a” and “an” shall mean “at least one” unless specified to the contrary or the context clearly indicates otherwise. Thus, embodiments using “a hydrogenating metal” include embodiments where one, two or more hydrogenating metals are used, unless specified to the contrary or the context clearly indicates that only one hydrogenating metal is used.

As used herein, the term “cyclohexylbenzene” shall mean benzene substituted by a single cyclohexyl group, unless specified to the contrary or the context clearly indicates otherwise. As used herein, the generic term “dicyclohexylbenzene” shall include 1,2-dicyclohexylbenzene, 1,3-dicyclohexylbenzene, 1,4-dicyclohexylbenzene, and mixtures and combinations of at least two thereof in any proportion. As used herein, the generic term “tricyclohexylbenzene” shall include 1,2,3-tricyclohexylbenzene, 1,2,4-tricyclohexylbenzene and 1,3,5-tricyclohexylbenzene, and combinations and mixtures thereof at any proportion. The generic term “polycycloyhexylbenzene” shall include any of the dicyclohexylbenzene isomers and tricyclohexylbenzene isomers described above, and combinations and mixtures of at least two thereof in any proportion.

Described herein is a process for producing a mixture of cyclohexanone and cyclohexanol suitable for use as KA oil. The process comprises catalytic hydrogenation of a feed comprising cyclohexanone and phenol so as to convert at least part of the cyclohexanone and/or phenol in the feed into cyclohexanol and thereby produce a hydrogenation product containing cyclohexanone and cyclohexanol. The desired cyclohexanone/cyclohexanol mixture can then be obtained from the hydrogenation product, for example by distillation.

A suitable feed for the present process can be produced by the acid-catalyzed cleavage of cyclohexylbenzene hydroperoxide as part of an integrated process for producing phenol and/or cyclohexanone from benzene. In such a process benzene is initially alkylated or hydroalkylated to produce cyclohexylbenzene and the cyclohexylbenzene is oxidized to produce cyclohexylbenzene hydroperoxide. The ensuing description will focus on this integrated process.

### Production of Cyclohexylbenzene

The first step in the integrated process is the production of cyclohexylbenzene by the alkylation of benzene with cyclohexene according to the following reaction:

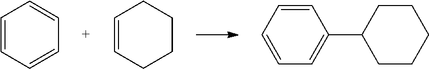 [SMILES: c1ccccc1 ; C1=CCCCC1 ; c1ccc(C2CCCCC2)cc1]

The cyclohexene can be supplied to the reaction as a separate feed from the benzene, but normally is produced in situ by the selective hydrogenation of benzene in the presence of a bifunctional catalyst. Such a reaction is generally termed “hydroalkylation” and may be summarized as follows:

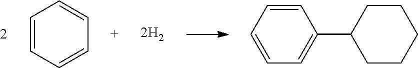 [SMILES: c1ccccc1 ; c1ccc(C2CCCCC2)cc1]

Any commercially available benzene feed can be used in the hydroalkylation step, but preferably the benzene has a purity level of at least 99 wt %. Similarly, although the source and purity of hydrogen is not critical, it is desirable that the hydrogen is at least 99 wt % pure.

The total feed to the hydroalkylation step may contain less than 1000 ppm, such as less than 500 ppm, for example less than 100 ppm, water. In addition, the total feed may contain less than 100 ppm, such as less than 30 ppm, for example less than 3 ppm, sulfur and less than 10 ppm, such as less than 1 ppm, for example less than 0.1 ppm, nitrogen.

Hydrogen can be supplied to the hydroalkylation step over a wide range of values, but the hydrogen supply is desirably arranged such that the molar ratio of hydrogen to benzene in the hydroalkylation feed is from about 0.15:1 to about 15:1, such as from about 0.4:1 to about 4:1, for example from about 0.4 to about 0.9:1.

In addition to the benzene and hydrogen, a diluent, which is substantially inert under hydroalkylation conditions, may be supplied to the hydroalkylation reaction. The diluent may be a hydrocarbon, in which the desired cycloalkylaromatic product, in this case cyclohexylbenzene, is soluble, such as a straight chain paraffinic hydrocarbon, a branched chain paraffinic hydrocarbon, and/or a cyclic paraffinic hydrocarbon. Examples of suitable diluents are decane and cyclohexane. Cyclohexane is a particularly attractive diluent since it is an unwanted by-product of the hydroalkylation reaction.

Although the amount of diluent is not narrowly defined, advantageously the diluent is added in an amount such that the weight ratio of the diluent to the aromatic compound is at least 1:100; for example at least 1:10, but no more than 10:1, for example no more than 4:1.

The hydroalkylation reaction can be conducted in a wide range of reactor configurations including fixed bed, slurry reactors, and/or catalytic distillation towers. In addition, the hydroalkylation reaction can be conducted in a single reaction zone or in a plurality of reaction zones, in which at least the hydrogen is introduced to the reaction in stages. Suitable reaction temperatures are from about 100° C. to about 400° C., such as from about 125° C. to about 250° C., while suitable reaction pressures are from about 100 kPa to about 7,000 kPa, such as from about 500 kPa to about 5,000 kPa.

The catalyst employed in the hydroalkylation reaction is a bifunctional catalyst comprising a hydrogenating metal component and an alkylating solid acid component. Advantageously, the alkylating solid acid component comprises a molecular sieve of the MCM-22 family. The term “MCM-22 family material” (or “material of the MCM-22 family” or “molecular sieve of the MCM-22 family”), as used herein, includes one or more of:
molecular sieves made from a common first degree crystalline building block unit cell, which unit cell has the MWW framework topology. (A unit cell is a spatial arrangement of atoms which if tiled in three-dimensional space describes the crystal structure. Such crystal structures are discussed in the “Atlas of Zeolite Framework Types”, Fifth edition, 2001, the entire content of which is incorporated as reference);
molecular sieves made from a common second degree building block, being a 2-dimensional tiling of such MWW framework topology unit cells, forming a monolayer of one unit cell thickness, preferably one c-unit cell thickness;
molecular sieves made from common second degree building blocks, being layers of one or more than one unit cell thickness, wherein the layer of more than one unit cell thickness is made from stacking, packing, or binding at least two monolayers of one unit cell thickness. The stacking of such second degree building blocks can be in a regular fashion, an irregular fashion, a random fashion, or any combination thereof and
molecular sieves made by any regular or random 2-dimensional or 3-dimensional combination of unit cells having the MWW framework topology.

Molecular sieves of MCM-22 family generally have an X-ray diffraction pattern including d-spacing maxima at 12.4±0.25, 6.9±0.15, 3.57±0.07 and 3.42±0.07 Angstrom. The X-ray diffraction data used to characterize the material are obtained by standard techniques using the K-alpha doublet of copper as the incident radiation and a diffractometer equipped with a scintillation counter and an associated computer as the collection system. Molecular sieves of MCM-22 family include MCM-22 (described in U.S. Pat. No. 4,954,325), PSH-3 (described in U.S. Pat. No. 4,439,409), SSZ-25 (described in U.S. Pat. No. 4,826,667), ERB-1 (described in European Patent No. 0293032), ITQ-1 (described in U.S. Pat. No. 6,077,498), ITQ-2 (described in International Patent Publication No. WO97/17290), MCM-36 (described in U.S. Pat. No. 5,250,277), MCM-49 (described in U.S. Pat. No. 5,236,575), MCM-56 (described in U.S. Pat. No. 5,362,697), and mixtures and combinations thereof. Molecular sieves similar to MCM-22 family materials, such as UZM-8 (described in U.S. Pat. No. 6,756,030), may be used alone or together with the MCM-22 family materials. Desirably, the molecular sieve is selected from (a) MCM-49; (b) MCM-56; and (c) isotypes of MCM-49 and MCM-56, such as ITQ-2.

Any known hydrogenating metal can be employed in the hydroalkylation catalyst, although suitable metals include palladium, ruthenium, nickel, zinc, tin, and cobalt, with palladium being particularly advantageous. Desirably, the amount of hydrogenating metal present in the catalyst is from about 0.05 to about 10 wt %, such as from about 0.1 to about 5 wt %, of the catalyst. Where the MCM-22 family molecular sieve is an aluminosilicate, the amount of hydrogenating metal present may be such that the molar ratio of the aluminum in the molecular sieve to the hydrogenating metal is from about 1.5 to about 1500, for example from about 75 to about 750, such as from about 100 to about 300.

The hydrogenating metal may be directly supported on the MCM-22 family molecular sieve by, for example, impregnation or ion exchange. Preferably, however, at least 50 wt %, for example at least 75 wt %, and desirably substantially all of the hydrogenating metal is supported on an inorganic oxide separate from but composited with the molecular sieve. In particular, it is found that by supporting the hydrogenating metal on the inorganic oxide, the activity of the catalyst and its selectivity to cyclohexylbenzene and dicyclohexylbenzene are increased as compared with an equivalent catalyst in which the hydrogenating metal is supported on the molecular sieve.

The inorganic oxide employed in such a composite hydroalkylation catalyst is not narrowly defined provided it is stable and inert under the conditions of the hydroalkylation reaction. Suitable inorganic oxides include oxides of Groups 2, 4, 13, and 14 of the Periodic Table of Elements, such as alumina, titania, and/or zirconia. As used herein, the numbering scheme for the Periodic Table Groups is as disclosed in Chemical and Engineering News, 63(5), 27 (1985).

The hydrogenating metal is deposited on the inorganic oxide, e.g., by impregnation, before the metal-containing inorganic oxide is composited with the molecular sieve. The catalyst composite may be produced by co-pelletization, in which a mixture of the molecular sieve and the metal-containing inorganic oxide are formed into pellets at high pressure (desirably about 350 kPa to about 350,000 kPa), or by co-extrusion, in which a slurry of the molecular sieve and the metal-containing inorganic oxide, optionally together with a separate binder, are forced through a die. If necessary, additional hydrogenating metal can subsequently be deposited on the resultant catalyst composite.

Although the hydroalkylation reaction using an MCM-22 family zeolite catalyst is highly selective towards cyclohexylbenzene, the effluent from the hydroalkylation reaction will inevitably contain some dicyclohexylbenzene by-product. Depending on the amount of this dicyclohexylbenzene, it may be desirable to either (a) transalkylate the dicyclohexylbenzene with additional benzene or (b) dealkylate the dicyclohexylbenzene to maximize the production of the desired monoalkylated species.

Transalkylation with additional benzene may be conducted in a transalkylation reactor, separate from the hydroalkylation reactor, over a suitable transalkylation catalyst, such as a molecular sieve of the MCM-22 family, zeolite beta, MCM-68 (see U.S. Pat. No. 6,049,018), zeolite Y and mordenite. The transalkylation reaction is desirably conducted under at least partial liquid phase conditions, which suitably include a temperature of about 100 to about 300° C., a pressure of about 800 to about 3500 kPa, a weight hourly space velocity of about 1 to about 10 hr⁻¹ on total feed, and a benzene/dicyclohexylbenzene weight ratio of about 1:1 to about 5:1.

Dealkylation or cracking may also be effected in a reactor separate from the hydroalkylation reactor, such as a reactive distillation unit, at a temperature of about 150° C. to about 500° C. and a pressure of 15 to 500 psig (200 to 3550 kPa) over an acid catalyst such as an aluminosilicate, an aluminophosphate, a silicoaluminophosphate, amorphous silica-alumina, an acidic clay, a mixed metal oxide, such as WOₓ/ZrO₂, phosphoric acid, sulfated zirconia and mixtures thereof. Desirably, the acid catalyst includes at least one aluminosilicate, aluminophosphate or silicoaluminophosphate of the FAU, AEL, AFI and MWW family. Unlike transalkylation, dealkylation can be conducted in the absence of added benzene, although it may be desirable to add benzene to the dealkylation reaction to reduce coke formation. In this case, the weight ratio of benzene to poly-alkylated aromatic compounds in the feed to the dealkylation reaction is desirably from 0 to about 0.9, such as from about 0.01 to about 0.5. Similarly, although the dealkylation reaction can be conducted in the absence of added hydrogen, hydrogen is advantageously introduced into the dealkylation reactor to assist in coke reduction. Suitable hydrogen addition rates are such that the molar ratio of hydrogen to poly-alkylated aromatic compound in the total feed to the dealkylation reactor is from about 0.01 to about 10.

Another significant by-product of the hydroalkylation reaction is cyclohexane. Although a C₆-rich stream comprising cyclohexane and unreacted benzene can be readily removed from the hydroalkylation reaction effluent by distillation, owing to the similarity in the boiling points of benzene and cyclohexane, the C₆-rich stream is difficult to further separate by simple distillation. However, some or all of the C₆-rich stream can be recycled to the hydroalkylation reactor to provide not only part of the benzene feed but also part of the diluents mentioned above.

In some cases, it may be desirable to supply some of the C₆-rich stream to a dehydrogenation reaction zone, where the C₆-rich stream is contacted with a dehydrogenation catalyst under dehydrogenation conditions sufficient to convert at least part of the cyclohexane in the C₆-rich stream portion to benzene, which again can be recycled to the hydroalkylation reaction. The dehydrogenation catalyst desirably comprises (a) a support; (b) a hydrogenation-dehydrogenation component and (c) an inorganic promoter. The support (a) may be selected from the group consisting of silica, a silicate, an aluminosilicate, zirconia, and carbon nanotubes, and preferably comprises silica. Suitable hydrogenation-dehydrogenation components (b) comprise at least one metal selected from Groups 6 to 10 of the Periodic Table of Elements, such as platinum, palladium and compounds and mixtures thereof. Desirably, the hydrogenation-dehydrogenation component is present in an amount from about 0.1 to about 10 wt % of the catalyst. A suitable inorganic promoter (c) comprises at least one metal or compound thereof selected from Group 1 of the Periodic Table of Elements, such as a potassium compound. The promoter may be present in an amount from about 0.1 to about 5 wt % of the catalyst. Suitable dehydrogenation conditions include a temperature of about 250° C. to about 500° C., a pressure of about atmospheric to about 500 psig (100 to 3550 kPa), a weight hourly space velocity of about 0.2 to 50 hr⁻¹, and a hydrogen to hydrocarbon feed molar ratio of about 0 to about 20.

Other disadvantageous impurities of the hydroalkylation reaction are bicyclohexyl (BCH) and the methylcyclopentylbenzene (MCPB) isomers which, because of the similarity in their boiling points, are difficult to separate from the desired cyclohexylbenzene by distillation. Moreover, although 1,2-methylcyclopentylbenzene (2-MCPB), and 1,3-methylcyclopentylbenzene (3-MCPB) are readily converted in the subsequent oxidation/cleavage steps to the phenol and methylcyclopentanones, which are valuable products, 1,1-methylcyclopentylbenzene (1-MCPB) is substantially inert to the oxidation step and so, if not removed, will build up in the C₁₂ stream. Similarly, bicyclohexyl (BCH) can lead to separation problems downstream. Thus, at least part of the hydroalkylation reaction product may be treated with a catalyst under conditions to remove at least 1,1-methylcyclopentylbenzene and/or bicyclohexyl from the product. The catalyst may be an acid catalyst, such as an aluminosilicate zeolite, and especially faujasite and the treatment is conducted at a temperature of about 100° C. to about 350° C., such as about 130° C. to about 250° C., for a time of about 0.1 to about 3 hours, such as about 0.1 to about 1 hours. The catalytic treatment is believed to isomerize the 1,1-methylcyclopentylbenzene (1-MCPB) to the more readily oxidizable 1,2-methylcyclopentylbenzene (2-MCPB), and 1,3-methylcyclopentylbenzene (3-MCPB). The bicyclohexyl is believed to react with benzene present in the hydroalkylation reaction product to produce cyclohexane and more of the desired cyclohexylbenzene according to the following reaction:

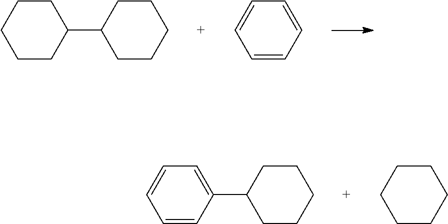 [SMILES: C1CCC(C2CCCCC2)CC1 ; c1ccccc1 ; c1ccc(C2CCCCC2)cc1 ; C1CCCCC1]

The catalytic treatment can be conducted on the direct product of the hydroalkylation reaction or after distillation of the hydroalkylation reaction product to separate the C₆ and/or the heavy fraction.

The cyclohexylbenzene product from the hydroalkylation reaction and any downstream reaction to remove the impurities discussed herein is separated from the reaction effluent(s) and is fed to the oxidation reaction described in more detail below.

### Cyclohexylbenzene Oxidation

In order to convert the cyclohexylbenzene into phenol and cyclohexanone, the cyclohexylbenzene is initially oxidized to the corresponding hydroperoxide, particularly cyclohexyl-1-phenyl-1-hydroperoxide. This is accomplished by contacting the cyclohexylbenzene with an oxygen-containing gas, such as air and various derivatives of air. For example, it is possible to use air that has been compressed and filtered to remove particulates, air that has been compressed and cooled to condense and remove water, or air that has been enriched in oxygen above the natural approximately 21 mol % in air through membrane enrichment of air, cryogenic separation of air or other conventional means.

The oxidation step can be conducted autogenously or more preferably in the presence of a catalyst. Although any catalyst can be employed, a preferred oxidation catalyst includes an N-hydroxy substituted cyclic imide described in U.S. Pat. No. 6,720,462, which is incorporated herein by reference in its entirety for this purpose. For example, N-hydroxyphthalimide (NHPI), 4-amino-N-hydroxyphthalimide, 3-amino-N-hydroxyphthalimide, tetrabromo-N-hydroxyphthalimide, tetrachloro-N-hydroxyphthalimide, N-hydroxyhetimide, N-hydroxyhimimide, N-hydroxytrimellitimide, N-hydroxybenzene-1,2,4-tricarboximide, N,N′-dihydroxy(pyromellitic diimide), N,N′-dihydroxy(benzophenone-3,3′,4,4′-tetracarboxylic diimide), N-hydroxymaleimide, pyridine-2,3-dicarboximide, N-hydroxysuccinimide, N-hydroxy(tartaric imide), N-hydroxy-5-norbornene-2,3-dicarboximide, exo-N-hydroxy-7-oxabicyclo[2.2.1]hept-5-ene-2,3-dicarboximide, N-hydroxy-cis-cyclohexane-1,2-dicarboximide, N-hydroxy-cis-4-cyclohexene-1,2 dicarboximide, N-hydroxynaphthalimide sodium salt or N-hydroxy-o-benzenedisulphonimide may be used. The catalyst may be N-hydroxyphthalimide. Another suitable catalyst is N,N′,N″-trihydroxyisocyanuric acid. Each of the above cyclic imide catalysts contain the heteroatom nitrogen.

These oxidation catalysts can be used either alone or in conjunction with a free radical initiator, and further can be used as liquid-phase, homogeneous catalysts or can be supported on a solid carrier to provide a heterogeneous catalyst. Desirably, the N-hydroxy substituted cyclic imide or the N,N′,N″-trihydroxyisocyanuric acid is employed in an amount from 0.0001 wt % to 15 wt %, such as from 0.001 wt % to 5 wt %, of the cyclohexylbenzene.

Suitable conditions for the oxidation step include a temperature from about 70° C. to about 200° C., such as about 90° C. to about 130° C., and a pressure of about 50 to 10,000 kPa. A basic buffering agent may be added to react with acidic by-products that may form during the oxidation. In addition, an aqueous phase may be introduced. The reaction can take place in a batch or continuous flow fashion.

The reactor used for the oxidation reaction may be any type of reactor that allows for introduction of oxygen to cyclohexylbenzene, and may further efficaciously provide contacting of oxygen and cyclohexylbenzene to effect the oxidation reaction. For example, the oxidation reactor may comprise a simple, largely open vessel with a distributor inlet for the oxygen-containing stream. The oxidation reactor may have means to withdraw and pump a portion of its contents through a suitable cooling device and return the cooled portion to the reactor, thereby managing the exothermicity of the oxidation reaction. Alternatively, cooling coils providing indirect cooling, say by cooling water, may be operated within the oxidation reactor to remove the generated heat. Alternatively, the oxidation reactor may comprise a plurality of reactors in series, each conducting a portion of the oxidation reaction, optionally operating at different conditions selected to enhance the oxidation reaction at the pertinent conversion range of cyclohexylbenzene or oxygen, or both, in each. The oxidation reactor may be operated in a batch, semi-batch, or continuous flow manner.

Desirably, the product of the cyclohexylbenzene oxidation reaction contains at least 5 wt %, such as at least 10 wt %, for example at least 15 wt %, or at least 20 wt % cyclohexyl-1-phenyl-1-hydroperoxide based upon the total weight of the oxidation reaction effluent. The oxidation reaction effluent may contain no greater than 80 wt %, or no greater than 60 wt %, or no greater than 40 wt %, or no greater than 30 wt %, or no greater than 25 wt % of cyclohexyl-1-phenyl-1-hydroperoxide based upon the total weight of the oxidation reaction effluent.

The oxidation reaction effluent may also comprise residual cyclohexylbenzene. For example, the oxidation reaction effluent may include residual cyclohexylbenzene in an amount of at least 50 wt %, or at least 60 wt %, or at least 65 wt %, or at least 70 wt %, or at least 80 wt %, or at least 90 wt %, based upon total weight of the oxidation reaction effluent.

### Treatment of the Oxidation Reaction Effluent

In addition to cyclohexylbenzene hydroperoxide and unreacted cyclohexylbenzene, the oxidation reaction effluent may also contain some of the cyclic imide used as a catalyst in the oxidation reaction. Since cyclic imides are expensive and can negatively affect downstream reactions, it is desirable to remove and/or obtain at least part of the cyclic imide from the oxidation reaction effluent for recycle back to the oxidation step. Removal of the cyclic imide may comprise contacting the oxidation reaction effluent with an aqueous solution of a base, particularly a weak base having a pKb value greater than or equal to the pKa of the cyclic imide of the first catalyst, whereby the imide is extracted into the aqueous phase, leaving an organic phase which comprises said oxidized hydrocarbon product and a reduced level of cyclic imide. Treatment of the oxidation effluent to remove at least part of the cyclic imide may comprise contacting the effluent with an effective solid sorbent, such as a metal oxide or a metal carbonate and/or hydrogen carbonate.

Prior to feeding the oxidative reaction effluent to the cleavage step, the effluent may be treated to increase the concentration of the cyclohexylbenzene hydroperoxide. Suitable concentration steps include fractional distillation to remove at least part of the cyclohexylbenzene and fractional crystallization to separate solid cyclohexylbenzene hydroperoxide from the oxidation reaction effluent. The concentration step(s) may be used to produce a cleavage feed containing at least 40 wt % and no greater than 95 wt %, for example from 60 wt % to 85 wt %, of cyclohexylbenzene hydroperoxide, and at least 5 wt % and at most 60 wt %, for example from 15 wt % to 40 wt %, of cyclohexylbenzene.

### Hydroperoxide Cleavage

The cleavage feed is then fed to one or more cleavage reactors, where the cyclohexylbenzene hydroperoxide in the cleavage feed undergoes liquid phase acid-catalyzed decomposition to produce phenol and cyclohexanone.

The acid catalyst used in the cleavage reaction may be at least partially soluble in the cleavage reaction mixture, is stable at a temperature of at least 185° C. and has a lower volatility (higher normal boiling point) than cyclohexylbenzene. Typically, the acid catalyst is also at least partially soluble in the cleavage reaction product. Suitable acid catalysts include, but are not limited to, Brønsted acids, Lewis acids, sulfonic acids, perchloric acid, phosphoric acid, hydrochloric acid, p-toluene sulfonic acid, aluminum chloride, oleum, sulfur trioxide, ferric chloride, boron trifluoride, sulfur dioxide and sulfur trioxide. Sulfuric acid is a preferred acid catalyst.

Using homogeneous cleavage catalysts, the cleavage reaction mixture contains at least 50 weight-parts-per-million (wppm) and no greater than 5000 wppm of the catalyst, or at least 100 wppm to no greater than 3000 wppm, or at least 150 wppm to no greater than 2000 wppm of the catalyst, or at least 300 wppm to no greater than 1500 wppm of the catalyst, based upon total weight of the cleavage reaction mixture.

A heterogeneous acid catalyst may be employed for the cleavage reaction, such as a molecular sieve, and in particular a molecular sieve having a pore size in excess of 7 [symbol] . Examples of suitable molecular sieves include zeolite beta, zeolite Y, zeolite X, ZSM-12 and mordenite. The molecular sieve may comprise a FAU type zeolite having a unit cell size less than 24.35 Å, such as less than or equal to 24.30 Å, even less than or equal to 24.25 Å. The zeolite can be used in unbound form or can be combined with a binder, such as silica or alumina, such that the overall catalyst (zeolite plus binder) comprises from about 20 wt % to about 80 wt % of the zeolite.

The cleavage reaction mixture may also contain a polar solvent, such as an alcohol containing less than 6 carbons, such as methanol, ethanol, iso-propanol, and/or ethylene glycol; a nitrile, such as acetonitrile and/or propionitrile; nitromethane; and a ketone containing 6 carbons or less such as acetone, methylethyl ketone, 2- or 3-pentanone, cyclohexanone, and methylcyclopentanone. The polar solvent may be phenol and/or cyclohexanone recycled from the cleavage product after cooling. Advantageously, the polar solvent is added to the cleavage reaction mixture such that the weight ratio of the polar solvent to the cyclohexylbenzene hydroperoxide in the mixture is in the range of about 1:100 to about 100:1, such as about 1:20 to about 10:1, and the mixture comprises about 5 wt % to about 40 wt % of the cyclohexylbenzene hydroperoxide. The addition of the polar solvent is found not only to increase the degree of conversion of the cyclohexylbenzene hydroperoxide in the cleavage reaction but also to increase the selectivity of the conversion to phenol and cyclohexanone. Although the mechanism is not fully understood, it is believed that the polar solvent reduces the free radical inducted conversion of the cyclohexylbenzene hydroperoxide to undesired products such as hexanophenone and phenylcyclohexanol.

The cleavage conditions are desirably selected so that the cleavage reaction mixture is completely or predominantly in the liquid phase during the cleavage reaction. The cleavage reaction may be conducted for a time sufficient to convert at least 50%, desirably at least 75%, of the cyclohexylbenzene hydroperoxide in the cleavage reaction mixture and produce a cleavage effluent containing phenol and cyclohexanone.

The reactor used to effect the cleavage reaction may be any type of reactor known to those skilled in the art. For example, the cleavage reactor may be a simple, largely open vessel operating in a near-continuous stirred tank reactor mode, or a simple, open length of pipe operating in a near-plug flow reactor mode. The cleavage reactor may comprise a plurality of reactors in series, each performing a portion of the conversion reaction, optionally operating in differential modes and at different conditions selected to enhance the cleavage reaction at the pertinent conversion range. The cleavage reactor may comprise a catalytic distillation unit.

The cleavage reactor may be operable to transport a portion of the contents through a cooling device and return the cooled portion to the cleavage reactor, thereby managing the exothermicity of the cleavage reaction. Alternatively, the reactor may be operated adiabatically. Cooling coils operating within the cleavage reactor(s) can be used to remove at least a portion of the heat generated.

The major products of the cleavage reaction are phenol and cyclohexanone, which are present in the cleavage effluent in substantially equimolar amounts normally together with residual cyclohexylbenzene and cyclohexylbenzene hydroperoxide and, sometimes, residual sulfuric acid catalyst.

### Production and Separation of KA Oil

On leaving the cleavage reactor, the cleavage effluent may be cooled and thereafter separated into a cleavage product stream, from which phenol and cyclohexanone can be recovered, and a cleavage recycle stream, which can be mixed with the cleavage feed. Separation of the cleavage recycle stream can be effected without prior modification of the composition of cleavage effluent so that the cleavage recycle stream comprises an aliquot of the cleavage effluent. The cleavage recycle stream has substantially the same composition as the cleavage effluent such that, e.g., each component has a concentration differential within 2 wt % or even within 1 wt % between the cleavage effluent and the cleavage recycle. Such differential may be due to additional reactions taking place in the cleavage effluent stream. The weight ratio of the cleavage recycle stream to the cleavage product stream may range from r1 to r2, where r1 and r2 can be, independently, 0.1, 0.2, 0.3, 0.4, 0.5, 0.6, 0.7, 0.8, 0.9, 1.0, 1.5, 2.0, 2.5, 3.0, 4.5, 5.0, 6.0, 7.0, 8.0, 9.0, 10, 12, 14, 15, 16, 18, 20, 25, 30, 35, 40, 45, 50, as long as r1<r2.

The cleavage product effluent, which can be a part or the entirety of the cleavage effluent, can be forwarded to a product recovery section, where at least a part of the cyclohexanone and phenol is recovered. However, as indicated above, cyclohexanone and phenol form an azeotropic mixture comprising about 28 wt % cyclohexanone and 72 wt % phenol. Thus, it is not possible to recover all of the cyclohexanone, or any of the phenol, from the cleavage effluent as a saleable product with reasonable purity by simple traditional distillation. Instead, an extractive solvent, such as diethylene glycol (DEG), may be added to the cleavage effluent to break the azeotrope, which will have to be removed in a subsequent step, adding to the complexity and cost of the separation. The present process can be advantageously used to minimize this problem by converting all or part of the cyclohexanone and phenol in the cleavage effluent to KA oil by catalytic hydrogenation. This approach reduces or avoids the need for complicated and costly separation, while at the same time satisfying the needs of the market for three separate products, namely, phenol, cyclohexanone, and KA oil. The KA oil product may comprise, e.g., at least 80 wt % (or at least 85 wt %, 90 wt %, 92 wt %, 93 wt %, 95 wt %, 96 wt %, 97 wt %, 98 wt %, 99 wt %, 99.5 wt %, 99.8 wt %, or even 99.9 wt %) of cyclohexanone and cyclohexanol in total, based on the total weight of the mixture.

Catalytic hydrogenation of part or all of the cleavage product effluent can be performed in any known hydrogenation reaction zone, such as one or more fixed bed reactors. Suitable hydrogenation catalysts may comprise at least one metal selected from Pd, Pt, Ru, Rh, Ir, O, Ni, Zn, Sn, Co, and compounds and mixtures thereof. Preferably, the catalyst comprises at least one precious metal such as Pt, Pd, Ru, Rh, Ir, and Os. More preferably, the catalyst comprises at least one of Pt and Pd. Still more preferably, the catalyst comprises both Pt and Pd, wherein the molar ratio of Pt to Pd can range from R1 to R2, where R1 and R2 can be, independently, 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10.0, as long as R1<R2. Alternatively, the molar ratio of Pt and Pd in the catalyst can be from R3 to R4, where R3 and R4 can be, independently, 0.5, 0.6, 0.7, 0.8, 0.9, 1.0, 2.0, 3.0, 4.0, 5.0, as long as R3<R4. As indicated in the examples below, a catalyst comprising both Pt and Pd can achieve a good balance of conversion of phenol and cyclohexanone and low selectivities towards light by-products such as cyclohexane and heavy by-products such as aldol condensation products and bicyclohexane ether. The metal(s) described above provides a least a part of the hydrogenation function of the catalyst. To that the end, the amount of the metal(s) based on the total weight of the catalyst can range from x1 wt % to x2 wt %, where x1 and x2 can be, independently, 0.01, 0.05, 0.10, 0.15, 0.20, 0.25, 0.30, 0.35, 0.35, 0.40, 0.45, 0.50, 0.55, 0.60, 0.65, 0.70, 0.75, 0.80, 0.85, 0.90, 0.95, 1.00, 1.50, 1.80, 2.00, 2.50, 3.00, 3.50, 4.00, 4.50, or 5.00, as long as x1<x2. It is highly desired that at least 80%, such as at least 85%, or at least 90%, or at least 95%, or at least 98%, or at least 99%, or even at least 99.5%, by mole, of the metal is in element state (i.e., having an oxidation state of zero) in the catalyst when in use.

In addition to the metal, the hydrogenation catalyst used in the process of the present disclosure may further comprise an inorganic oxide such as at least one of Al₂O₃, SiO₂, ZrO₂, MgO, CaO, Gd₂O₃, GeO₂, Y₂O₃, rare earth oxides, TiO₂, SnO, SnO₂, and combinations and mixtures thereof. The inorganic oxide may function as a support of the metal(s) mentioned above and/or a co-catalyst adjusting or enhancing the performance of the metal(s). Preferably, the hydrogenation catalyst comprises at least one of Al₂O₃, SiO₂. Alternatively, the hydrogenation catalyst comprises at least one of SnO and SnO₂. Alternatively, the hydrogenation catalyst comprises at least one of Al₂O₃ and SiO₂, and at least one of SnO and SnO₂. For example, the catalyst can comprise: (i) Al₂O₃ but no SiO₂, and SnO₂ but no SnO; (ii) Al₂O₃ but no SiO₂, and SnO but no SnO₂; (iii) Al₂O₃ but no SiO₂, and both SnO₂ and SnO; (iv) SiO₂ but no Al₂O₃, and SnO₂ but no SnO; (v) SiO₂ but no Al₂O₃, and SnO but no SnO₂; (vi) SiO₂ but no Al₂O₃, and both SnO₂ and SnO; (vii) both Al₂O₃ and SiO₂, and SnO₂ but no SnO; (viii) both Al₂O₃ and SiO₂, and SnO but no SnO₂; and (ix) Al₂O₃ and SiO₂, and both SnO₂ and SnO₂. The catalyst may comprise at least one of Pd and Pt, and at least one of SnO and SnO₂. Preferably, the catalyst comprises Pd, Pt, SnO, and SnO₂. Alternatively, the catalyst comprises Pt, Pd, at least one of Al₂O₃ and SiO₂, and at least one of SnO and SnO₂. Where the catalyst comprises both SnO and SnO₂, the molar ratio of SnO to SnO₂ can range from r1 to r2, where r1 and r2 can be, independently, 0.001, 0.01, 0.01, 1.00, 10.0, 100, 500, 800, 1000, as long as r1<r2. A catalyst may comprise SnO₂ at the beginning of the hydrogenation process. However due to the presence of hydrogen and the highly reducing atmosphere, part of the SnO₂ will be reduced to SnO in the hydrogenation process. Without intending to be bound by a particular theory, it is believed that the presence of both SnO and SnO₂ in the catalyst during the hydrogenation reaction is conducive to the hydrogenation process because this redox pair may participate in the electron transfer involving the metal and hydrogen, thereby aiding the hydrogenation reaction in desired directions. Preferably, the catalyst comprises Pd, Pt, SnO, SnO₂, and at least one of Al₂O₃ and SiO₂, each in amounts described above.

Suitable hydrogenation conditions comprise a temperature from T1° C. to T2° C. and an absolute pressure in a range from P1 kPa to P2 kPa, where T1 and T2 can be, independently, 10, 15, 20, 25, 30, 35, 40, 45, 50, 60, 70, 80, 90, 100, 120, 140, 150, 160, 180, 200, 220, 240, 250, 260, 280, or 300, as long as T1<T2; and P1 and P2 can be, independently, 100, 200, 300, 400, 500, 600, 700, 800, 900, 1000, 1500, 2000, 2500, 3000, 3500, 4000, 4500, 5000, 6000, 7000, 8000, 9000, 10,000, 11,000, 12,000, 13,000, or 14,000, as long as P1<P2. Catalytic hydrogenation converts part of the cyclohexanone in the cleavage effluent to cyclohexanol according to the reaction:

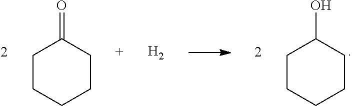 [SMILES: O=C1CCCCC1 ; "H2" ; OC1CCCCC1]

Where present in the hydrogenation feed, phenol can be, in whole or in part, converted to cyclohexanone and additional cyclohexanol according to the reaction:

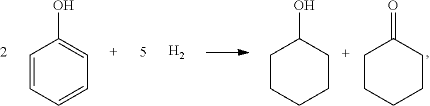

where the cyclohexanone produced can be further converted to cyclohexanol, as described above.

The product of the hydrogenation reaction may desirably comprise a mixture of cyclohexanone and cyclohexanol, which can readily be recovered from the hydrogenation product by, for example, fractionation. Pure cyclohexanone and cyclohexanol may be recovered from the hydrogenation product, if necessary. Preferably, a mixture product comprising both cyclohexanone and cyclohexanol is produced from the hydrogenation product. The mixture product can comprise c1 wt % to c2 wt % of cyclohexanone, balance essentially cyclohexanol, where c1 and c2 can be, independently, 10, 20, 30, 40, 50, 60, 70, 80, 90, as long as c1<c2. Preferably, the mixture product can comprise from 20% to 40% of cyclohexanone, and balance essentially cyclohexanol. Preferably, the mixture product may have a weight ratio of cyclohexanol to cyclohexanone of approximately 2. The mixture product comprising cyclohexanone and cyclohexanol can be advantageously used as KA oil for making high purity adipic acid by oxidation, which, in turn, is used for making Nylon-6,6.

Prior to hydrogenation, it may be desirable to feed at least part of the cleavage effluent to a fractionation system to recover a first fraction having a higher cyclohexanone concentration than the cleavage effluent and leave a second fraction having a lower cyclohexanone concentration as compared with the cleavage effluent. Provided the weight ratio of phenol to cyclohexanone in the fractionation feed is less than that in the phenol/cyclohexanone azeotrope, recovery of the cyclohexanone-rich first fraction can be achieved by simple distillation. Depending on the relative demand for cyclohexanone and KA oil, some or all of the cyclohexanone-rich first fraction can then be fed to the hydrogenation reaction to convert part of the cyclohexanone to cyclohexanol.

Prior to hydrogenation, it may be desirable to feed at least part of the cleavage effluent to a fractionation system to recover a first fraction having a higher phenol concentration than the cleavage effluent and leave a second fraction having a lower phenol concentration than the cleavage effluent. Depending on the relative demand for phenol, cyclohexanone and KA oil, some or all of the phenol-depleted second fraction and/or the phenol-rich first fraction can then be fed to the hydrogenation reaction to convert part of the cyclohexanone to cyclohexanol, and part or all of the phenol, to cyclohexanone and cyclohexanol.

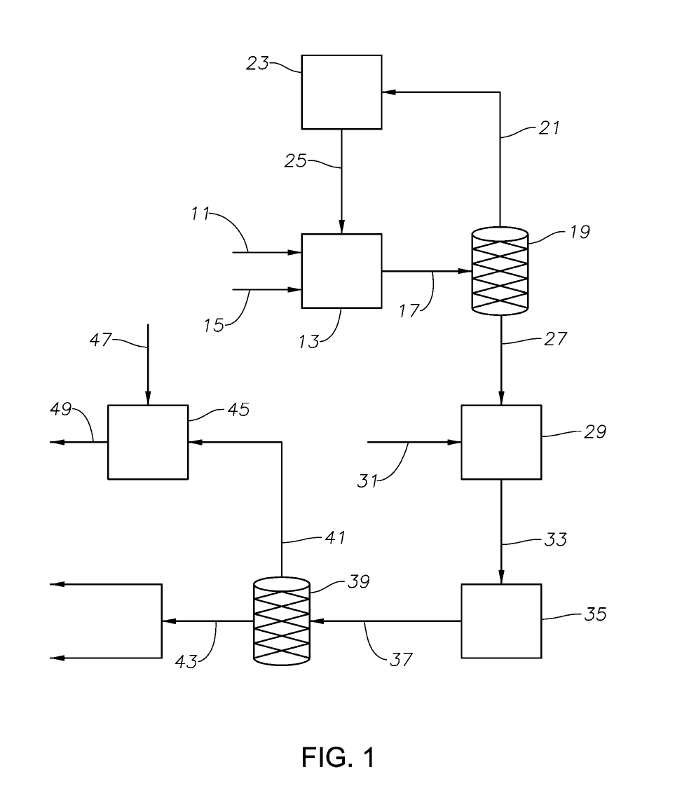

The invention will now be more particularly described with reference to FIG. 1, which is a flow diagram of a process for producing phenol, cyclohexanone and KA oil from benzene according to one example of the invention. In this process, benzene is supplied by line 11 to a hydroalkylation reactor 13, which also receives hydrogen from line 15. The conditions in the reactor 13 are such that part of the benzene reacts with the hydrogen in the presence of a hydroalkylation catalyst to produce a cyclohexylbenzene, which exits the reactor 13 as part of a hydroalkylation effluent in line 17.

The hydroalkylation effluent in line 17 also contains residual benzene, together with some cyclohexane by-product and is fed to a distillation unit 19, where a C₆-containing stream is removed from the hydroalkylation effluent in overhead line 21. The C₆-containing stream contains residual benzene and cyclohexane by-product in the hydroalkylation effluent and is fed to a dehydrogenation reactor 23, where the C₆-containing stream is contacted with a dehydrogenation catalyst under dehydrogenation conditions sufficient to convert at least part of the cyclohexane to additional benzene and produce a dehydrogenation effluent, which is recycled to the hydroalkylation reactor 13 by line 25.

The bottoms stream from the distillation unit 19, which contains most of the cyclohexylbenzene in the hydroalkylation effluent, is fed by line 27 to an oxidation reactor 29, which also receives an oxygen-containing gas from line 31. An oxidation catalyst, such as a cyclic imide, is contained in the reactor 29 and the conditions in the reactor 29 are such that at least part of the cyclohexylbenzene from line 27 is converted to cyclohexylbenzene hydroperoxide.

An effluent stream containing cyclohexylbenzene hydroperoxide is removed from the oxidation reactor 29 and fed by line 33 to a cleavage reactor 35, where at least part of the cyclohexylbenzene hydroperoxide is converted to phenol and cyclohexanone in the presence of an acid catalyst. The effluent from the cleavage reactor 35 is then fed by line 37 to a further distillation unit 39, where a fraction richer in cyclohexanone than the cleavage effluent is removed by line 41. The remainder of the cleavage effluent is then supplied by line 43 to a recovery system (not shown) for separation and purification of the phenol and remaining cyclohexanone.

The cyclohexanone-rich fraction in line 41 is fed to a hydrogenation reactor 45 which receives hydrogen via line 47 and contains a hydrogenation catalyst under conditions such that part of the cyclohexanone from line 41 is converted to cyclohexanol. The product of the hydrogenation process in reactor 45 is a mixture of cyclohexanone and cyclohexanol, which is fed by line 49 to a KA oil recovery system (not shown).

### EXAMPLES

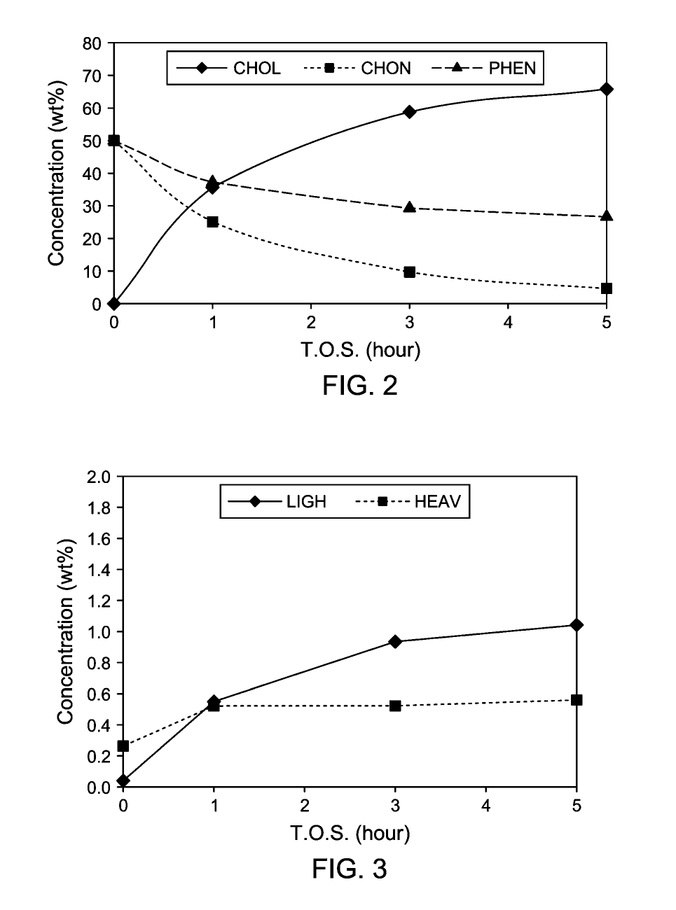

The invention will now be more particularly described with reference to the following non-limiting Examples and FIGS. 2-8 of the accompanying drawings. In the Examples, all percentages are by weight unless otherwise specified. The abbreviations used in the examples and figures are as follows:

CHON: cyclohexanone

PHEN: phenol

CHOL: cyclohexanol

LIGH: light by-products

HEAV: heavy by-products

T.O.S.: Time on stream

The light by-products include those having boiling points lower than cyclohexanone, cyclohexanol and phenol, such as cyclohexane and benzene. The heavy by-products include those having boiling points higher than phenol, cyclohexanone and cyclohexanol, such as cyclohexylbenzene, aldol condensation products and bicyclohexane ether. The concentrations of Pt, Pd, and Sn in the examples are calculated percentages by weight of the metals (i.e., not oxides, chloride or other source materials) based on the total weight of the final catalyst. The amounts of Pt, Pd, and Sn are calculated from the amounts of source materials (e.g., various salts) used in the preparation process. Actual measurement of the final catalyst after calcination showed high degree of congruence with the calculated concentrations.

### Example 1

### Preparation of 1.0% Pt/Al₂O₃ Catalyst

A Pt-containing catalyst was prepared using, as a catalyst support, an Al₂O₃ extrudate having a surface area of 306 m²/g, a pore volume of 0.85 cm³/g, and a pore size of 73 [symbol] , measured by BET N₂ adsorption. Pt was added to the Al₂O₃ support by impregnating the support with an aqueous solution of (NH₃)₄Pt(NO₃)₂. The Pt metal loading on the support was adjusted to 1.0%. After impregnation, the sample was placed in a glass dish at room temperature for 60 minutes to reach equilibrium. Then it was dried in air at 250° F. (120° C.) for 4 hours and subsequently calcined in a box furnace at 680° F. (360° C.) in air for 3 hours. The furnace was ramped at 3° F./minute to the calcination temperature and the air flow rate for the calcination was adjusted to 5 volumes per catalyst volume per minute.

### Example 2

### Preparation of 1.0% Pd/Al₂O₃ Catalyst

The preparation of Example 1 was repeated but with the Al₂O₃ support being impregnated with an aqueous solution of tetraammine palladium nitrate ((NH₃)₄Pd(NO₃)₂). The Pd metal loading on the support was adjusted to 1.0%.

### Example 3

### Preparation of 1.0% Pd/SiO₂Catalyst

Pd-containing 1/20″ (1.27 mm) quadrulobe silica extrudates were prepared by incipient wetness impregnation with aqueous solution of tetraammine palladium nitrate ((NH₃)₄Pd(NO₃)₂). The Pd metal loading on the support was adjusted to 1.0%. After impregnation, the sample was placed in a glass dish at room temperature for 60 minutes to reach equilibrium. Then it was dried in air at 250° F. (120° C.) for 4 hours and subsequently calcined in a box furnace at 680° F. (360° C.) in air for 3 hours. The furnace was ramped at 3° F./minute to the calcination temperature and the air flow rate for the calcination was adjusted to 5 volumes per catalyst volume per minute.

### Example 4-6

### Preparation of Catalysts Containing Pd and Sn Based on Al₂O₃ Support

The Al₂O₃ support used in Examples 1 and 2 was subjected to sequential impregnations with Sn and Pd. SnCl₂ was first added to the Al₂O₃ support by impregnation with an aqueous solution of SnCl₂. After the SnCl₂ impregnation, the sample was dried in air at 120° C. for 4 hours. Pd was then added to the Al₂O₃ support by impregnation with an aqueous solution of tetraammine palladium nitrate ((NH₃)₄Pd(NO₃)₂). After the Pd impregnation, the sample was dried in air at 120° C. for 4 hours and then calcined at 360° C. in air for 3 hours. The final catalyst contained 1.0% Pd and 0.30% Sn as tin oxide.

In the same manner, two additional catalysts with the following compositions were prepared:

Example 5: 1.0% Pd+0.15% Sn on Al₂O₃ support; and

Example 6: 0.5% Pd+0.30% Sn on Al₂O₃ support.

### Examples 7-9

### Preparation of Catalysts Containing Pt, Pd, and Sn Based on Al₂O₃ Support

Al₂O₃ extrudate used in Examples 1 and 2 was employed to prepare a supported catalyst containing Pd, Pt, and Sn. The catalyst was prepared by sequential impregnations. SnCl₂ was added to the Al₂O₃ support by impregnation with an aqueous solution of SnCl₂. After the Sn impregnation, the sample was dried in air at 120° C. for 4 hours. Pd and Pt were then added to Sn-containing Al₂O₃ support by co-impregnation with aqueous solutions of tetraammine palladium nitrate (NH₃)₄Pd(NO₃)₂, and tetraammine platinum nitrate (NH₃)₄Pt(NO₃)₂. The Pd and Pt metal loadings on the supports are 0.5 wt % Pd and 0.5% wt Pt, respectively. After Pd and Pt impregnation, the sample was dried in air at 120° C. for 4 hours, and then calcined at 360° C. in air for 3 hours. The final catalyst contained 0.5% Pd, 0.5% Pt and 0.30% Sn as tin oxide (Example 7).

In the same manner, two additional catalysts with the following compositions were prepared:

Example 8: 0.75% Pd+0.25% Pt+0.30% Sn on Al₂O₃ support; and

Example 9: 0.25% Pd+0.75% Pt+0.30% Sn on Al₂O₃ support.

### Example 10

### Hydrogenation of Phenol/Cyclohexanone Mixture with Catalyst of Example 1

In a 300-mL parr autoclave, 150.0 g blend of 50% phenol and 50% cyclohexanone and 4 g of the 1.0% Pt/Al₂O₃ catalyst of Example 1 were loaded. The autoclave was pressurized with hydrogen at a gauge pressure of 1000 psi (6895 kPa) and heated to 150° C. with stirring (400 rpm). The autoclave was then allowed to cool down to room temperature and the contents collected as the hydrogenation product. The concentrations of cyclohexanone, phenol and cyclohexanol with time on stream (T.O.S.) are measured. The results are shown in FIGS. 2 and 3.

### Example 11

### Hydrogenation of Phenol/Cyclohexanone Mixture with Catalyst of Example 2

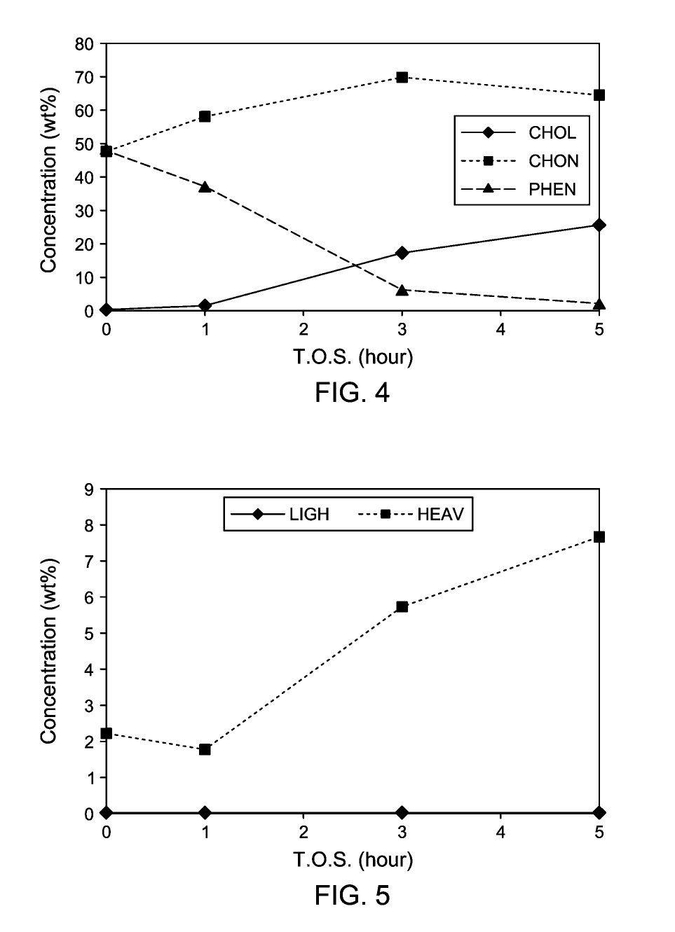

The process of Example 10 was repeated with the 1.0% Pd/Al₂O₃ catalyst of Example 2 and the results are shown in FIGS. 4 and 5.

Comparing FIG. 2 and FIG. 4, it will be seen that the Pd-containing catalyst of Example 2 is more reactive than the Pt-containing catalyst of Example 1 in phenol hydrogenation. However, the Pt-containing catalyst of Example 1 is more reactive than the Pd-containing catalyst of Example 2 in cyclohexanone hydrogenation.

Comparing FIG. 3 and FIG. 5, it will be seen that the production of heavy by-products is higher with the Pd-containing catalyst of Example 2 than with the Pt-containing catalyst of Example 1. However, the Pd-containing catalyst of Example 2 showed no measurable production of light by-products, whereas the Pt-containing catalyst of Example 1 was producing about 1% of light products at the end of the five hour test.

### Example 12

### Hydrogenation of Phenol/Cyclohexanone Mixture with Catalysts of Examples 2 and 3

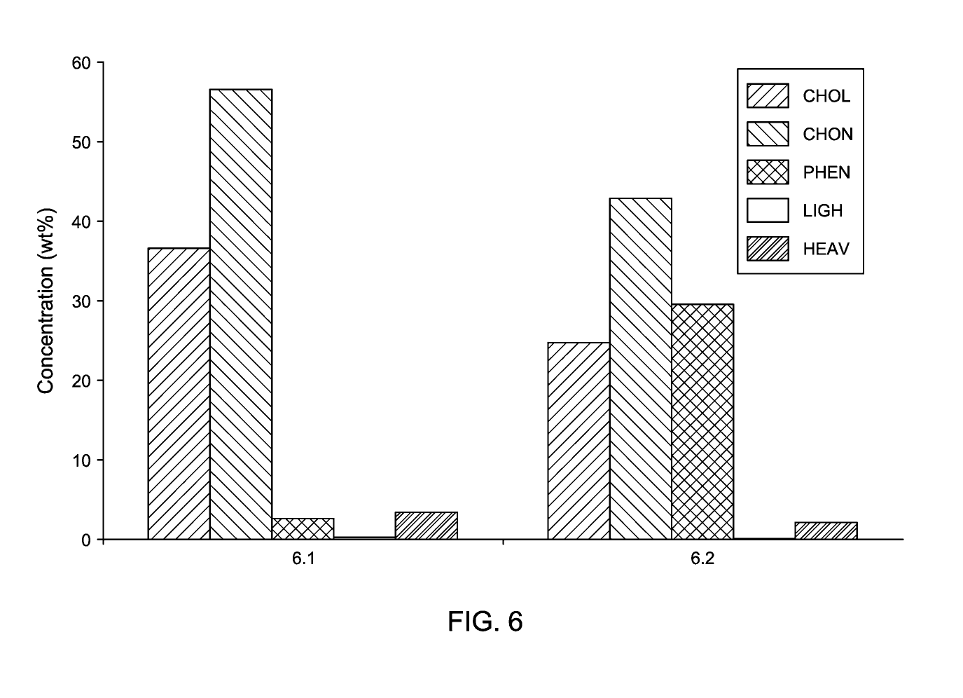

The process of Example 10 was repeated with the 1.0% Pd/Al₂O₃ catalyst of Example 2 and the 1.0% Pd/SiO₂ catalyst of Example 3. The results are compared in FIG. 6, which shows that the Pd/Al₂O₃ catalyst of Example 2 (bar group 6.1) is more reactive than the Pd/SiO₂ catalyst of Example 3 (bar group 6.2).

### Example 13

### Hydrogenation of Phenol/Cyclohexanone Mixture with Catalysts of Examples 2, 4, and 5

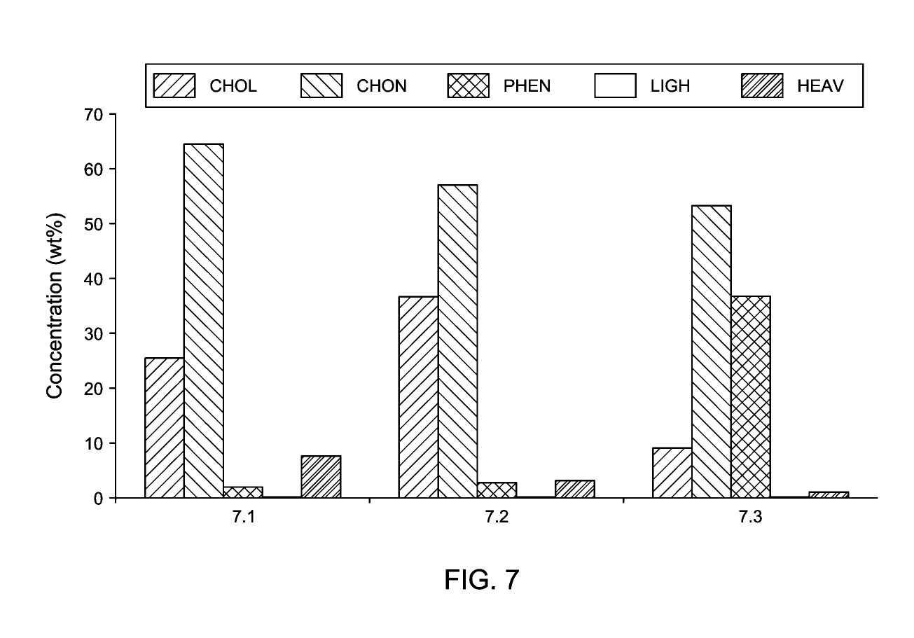

The process of Example 10 was repeated with the 1.0% Pd/Al₂O₃ catalyst of Example 2, the 1.0% Pd+0.30% Sn/Al₂O₃ catalyst of Example 4 and the 1.0% Pd+0.15% Sn/Al₂O₃ catalyst of Example 5. The results are compared in FIG. 7, where bar group 7.1 corresponds to Example 2, bar group 7.2 to Example 4, and bar group 7.3 to Example 5. FIG. 7 indicates that addition of Sn decreases selectivity to heavy by-products. However higher amounts (>0.15%) of Sn resulted in lower catalyst activity for phenol and cyclohexanone conversion.

### Example 14

### Hydrogenation of Phenol/Cyclohexanone Mixture with x % Pt/y % Pd/0.15% Sn/Alumina Catalysts

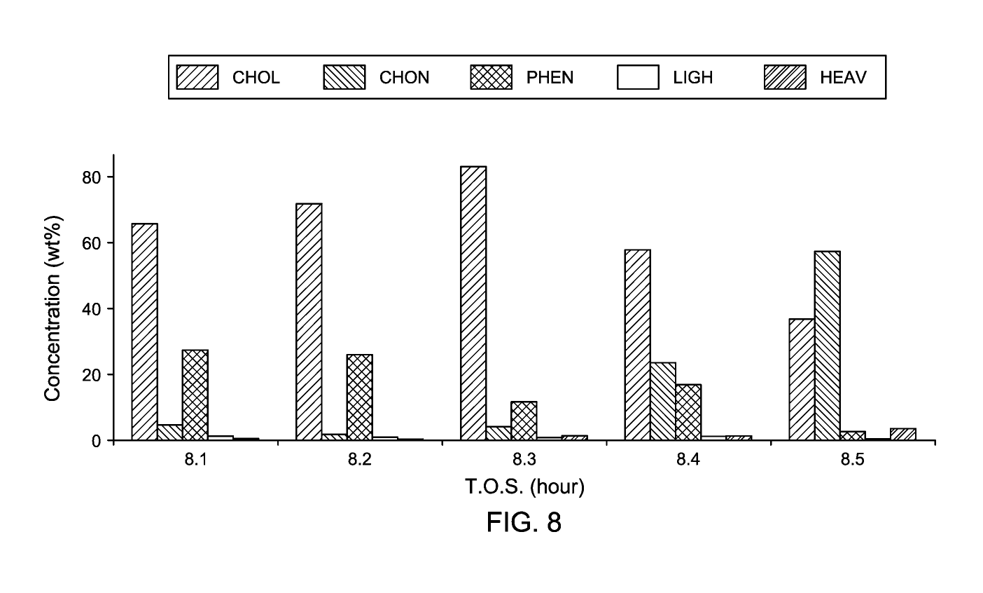

The process of Example 10 was repeated with a series of catalysts. The results are compared in FIG. 8, where the bar groups correspond to the catalysts with the following compositions:

TABLE I

| | | Catalyst |
|---|---|---|
| Bar | | Example |
| Group | Catalyst Composition | No. |
| 8.1 | 1.0% Pt on Al₂O₃ | 1 |
| 8.2 | 0.75% Pt + 0.25% Pd + 0.30% Sn on Al₂O₃ | 9 |
| 8.3 | 0.5% Pt + 0.5% Pd + 0.30% Sn on Al₂O₃ | 7 |
| 8.4 | 0.25% Pt + 0.75% Pd + 0.30% Sn on Al₂O₃ | 8 |
| 8.5 | 1.0% Pd on Al₂O₃ | 2 |

It is clear from FIG. 8 that the catalyst of Example 8 (0.5% Pt+0.5% Pd+0.30% Sn over Al₂O₃) exhibited the highest conversion and selectivity.

While the present invention has been described and illustrated by reference to particular embodiments, those of ordinary skill in the art will appreciate that the invention lends itself to variations not necessarily illustrated herein. For this reason, then, reference should be made solely to the appended claims for purposes of determining the true scope of the present invention.

Non-limiting aspects and/or embodiments of the present disclosure include:

E1. A process for producing a mixture of cyclohexanone and cyclohexanol, the process comprising:

(a) contacting a feed comprising cyclohexanone and phenol with hydrogen in the presence of a hydrogenation catalyst under hydrogenation conditions effective to convert at least part of the cyclohexanone and/or phenol in the feed into cyclohexanol and thereby produce a hydrogenation product containing cyclohexanone and cyclohexanol.

E1A. The process of E1, further comprising:

(b) obtaining a mixture comprising at least 90 wt % of cyclohexanone and cyclohexanol, based on the total weight of the mixture, from the hydrogenation product.

E2. The process of E1 or E1A, wherein the weight ratio of cyclohexanone to phenol in the feed is from 0.1 to 10.0.

E3. The process of any one of E1 to E2, wherein the hydrogenation catalyst comprises at least one of palladium, platinum, ruthenium, rhodium, nickel, zinc, tin, cobalt and compounds and mixtures thereof.

E4. The process of E3, wherein the hydrogenation catalyst comprises both platinum and palladium.

E5. The process of E4, wherein the hydrogenation catalyst comprise platinum and palladium at a molar ratio from 0.1 to 10.0.

E6. The process of any one of E3 to E5, wherein the hydrogenation catalyst further comprises at least one of the following inorganic oxides: Al₂O₃, SiO₂, ZrO₂, MgO, CaO, Gd2O3, GeO2, Y2O3, rare earth oxides, TiO2, SnO, SnO₂, and combinations and mixtures thereof.

E7. The process of E6, wherein the hydrogenation catalyst comprises at last two of Al₂O₃, SiO₂ and SnO₂.

E8. The process of E7, wherein the hydrogenation catalyst comprises at least one of SnO and SnO₂ and at least one of SiO₂ and Al₂O₃.

E9. The process of any one of E1 to E8, wherein the hydrogenation conditions comprise a temperature from 25° C. to 300° C. and an absolute pressure from 100 to 14,000 kPa.

E10. A process for producing a mixture of cyclohexanone and cyclohexanol, the process comprising:

(a) cleaving a cleavage feed comprising cyclohexylbenzene hydroperoxide to produce a cleavage effluent comprising phenol and cyclohexanone;

(b) obtaining a hydrogenation feed comprising phenol and/or cyclohexanone from at least a portion of the cleavage effluent; and

(c) contacting the hydrogenation feed with hydrogen in the presence of a hydrogenation catalyst in a hydrogenation reaction zone under hydrogenation conditions effective to convert at least part of the phenol and/or cyclohexanone in the portion of the hydrogenation feed into cyclohexanol and thereby produce a hydrogenation product containing cyclohexanone and cyclohexanol.

E10A. The process of E10, further comprising:

(d) obtaining a mixture comprising at least 90 wt % of cyclohexanone and cyclohexanol, based on the total weight of the mixture, from the hydrogenation product.

E11. The process of E10 or E10A, wherein the cleaving step (a) comprises contacting the cyclohexylbenzene hydroperoxide with an acid catalyst.

E12. The process of any one of E10 to E11, wherein the obtaining step (b) comprises:

(b1) obtaining from the cleavage effluent a first fraction having a higher cyclohexanone concentration than the cleavage effluent and a second fraction have a lower cyclohexanone concentration than the cleavage effluent; and

(b2) forming the hydrogenation feed from at least a portion of the first fraction.

E12A. The process of E12, wherein the obtaining step (b1) comprises distillation.

E13. The process of any one of E5 to E12A, wherein the hydrogenation catalyst in step (c) is selected from the group consisting of palladium, platinum, ruthenium, rhodium nickel, zinc, tin, cobalt and compounds and mixtures thereof

E14. The process of E13, wherein the hydrogenation catalyst comprises both platinum and palladium.

E15. The process of E14, wherein the hydrogenation catalyst comprises platinum and palladium at a molar ratio from 0.1 to 10.0.

E16. The process of any one of E10 to E15, wherein the hydrogenation catalyst further comprises at least one of the following inorganic oxides: Al₂O₃, SiO₂, ZrO₂, MgO, CaO, Gd₂O₃, GeO₂, Y₂O₃, rare earth oxides, TiO₂, SnO, SnO₂, and combinations and mixtures thereof.

E17. The process of E16, wherein the hydrogenation catalyst comprises Al₂O₃ and/or SiO₂.

E18. The process of E16, wherein the hydrogenation catalyst comprises at least two of Al₂O₃, SiO₂, and SnO₂.

E19. The process of E16, wherein the hydrogenation catalyst comprises at least one of SnO and SnO₂ and at least one of SiO₂ and Al₂O₃.

E20. The process of any one of E10 to E19, wherein the hydrogenation conditions in step (b) comprise a temperature from 25° C. to 300° C. and an absolute pressure from 100 to 14,000 kPa.

E21. A process for producing a mixture of cyclohexanone and cyclohexanol, the process comprising:

(a) contacting benzene and hydrogen with a hydroalkylation catalyst under hydroalkylation conditions to produce cyclohexylbenzene;

(b) oxidizing at least part of the cyclohexylbenzene from step (a) to produce an oxidation effluent comprising cyclohexylbenzene hydroperoxide;

(c) cleaving at least part of the cyclohexylbenzene hydroperoxide from step (b) to produce a cleavage effluent comprising phenol and cyclohexanone;

(d) obtaining a hydrogenation feed comprising phenol and/or cyclohexanone from at least a portion of the cleavage effluent; and

(e) contacting at least a portion of the hydrogenation feed with hydrogen in the presence of a hydrogenation catalyst in a hydrogenation reaction zone under hydrogenation conditions effective to convert at least part of the phenol and/or cyclohexanone in the portion of the hydrogenation feed into cyclohexanol and thereby produce a hydrogenation product containing cyclohexanone and cyclohexanol.

E21A. The process of E21, further comprising:

(f) obtaining a mixture comprising at least 90 wt % of cyclohexanone and cyclohexanol from the hydrogenation product.

E22. The process of E21 or E21A, wherein the hydroalkylation catalyst comprises a molecular sieve and a hydrogenation component.

E23. The process of any one of E21 to E22, wherein the molecular sieve comprises a molecular sieve of the MCM-22 family.

E24. The process of any one of E21 to E23, wherein the oxidizing step (b) comprises contacting at least part of the cyclohexylbenzene from step (a) with an oxygen-containing gas in the presence of an oxidation catalyst.

E25. The process of any one of E21 to E24, wherein the obtaining step (d) comprises:

(d1) obtaining from the cleavage effluent a first fraction having a higher cyclohexanone concentration than the cleavage effluent and a second fraction having a lower cyclohexanone concentration than the cleavage effluent; and

(d2) forming the hydrogenation feed from at least a portion of the first fraction.

E26. The process of E24, wherein the obtaining step (d1) comprises distillation.

E27. The process of any one of E21 to E26, wherein the hydrogenation catalyst in step (d) is selected from the group consisting of palladium, platinum, ruthenium, rhodium nickel, zinc, tin, cobalt and compounds and mixtures thereof

E28. The process of E27, wherein the hydrogenation catalyst comprises both platinum and palladium.

E29. The process of E28, wherein the hydrogenation catalyst comprises platinum and palladium at a molar ratio from 0.1 to 10.0.

E30. The process of any one of E27 to E29, wherein the hydrogenation catalyst further comprises at least one of the following inorganic oxides: Al₂O₃, SiO₂, ZrO₂, MgO, CaO, Gd₂O₃, GeO₂, Y₂O₃, rare earth oxides, TiO₂, SnO, SnO₂, and combinations and mixtures thereof.

E31. The process of E30, wherein the hydrogenation catalyst comprises Al₂O₃ and/or SiO₂.

E32. The process of E30, wherein the hydrogenation catalyst comprises at least two of Al₂O₃, SiO₂ and SnO₂.

E33. The process of E32, wherein the hydrogenation catalyst comprises at least one of SnO and SnO₂ and at least one of SiO₂ and Al₂O₃.

E34. The process of any one of E21 to E33, wherein the hydrogenation conditions in step (d) comprise a temperature from 25° C. to 300° C. and an absolute pressure from 100 to 14,000 kPa.

E35. A hydrogenation catalyst comprising platinum, palladium, tinand an inorganic oxide material.

E36. A hydrogenation catalyst of E35, wherein the molar ratio of platinum to palladium in the catalyst is from 0.1 to 10.0.

E37. A hydrogenation catalyst of E35 or E36, wherein the inorganic oxide material comprises at least one of Al₂O₃, SiO₂, ZrO₂, MgO, CaO, Gd₂O₃, GeO₂, Y₂O₃, rare earth oxides, TiO₂, SnO, SnO₂, and combinations and mixtures thereof.

E38. A hydrogenation catalyst of any one of E35 to E37, wherein the inorganic oxide material comprises SiO₂ and/or Al₂O₃.

E39. A hydrogenation catalyst of E37, wherein the inorganic oxide material comprises at least one of Al₂O₃ and SiO₂, and at least one of SnO and SnO₂.

E40. A hydrogenation catalyst of E39, wherein the inorganic oxide material comprises both SnO and SnO₂, and the molar ratio of SnO to SnO₂ is from 0.001 to 1000.

E41. A hydrogenation catalyst of any one of E35 to E40, wherein the total concentration of platinum and palladium is from 0.001 wt % to 5.0 wt %, based on the total weight of the catalyst.

E42. A hydrogenation catalyst of E40, wherein the total concentration of SnO and SnO₂ is from 0.01 wt % to 10.0 wt %, based on the total weight of the catalyst.

E43. A hydrogenation catalyst of any one of E35 to E42, wherein at least 95 wt % of platinum and palladium is at an oxidation state of zero.

1. A process for producing a mixture of cyclohexanone and cyclohexanol, the process comprising:
(a) contacting a feed comprising cyclohexanone and phenol with hydrogen in the presence of a hydrogenation catalyst under hydrogenation conditions effective to convert at least part of the cyclohexanone and/or phenol in the feed into cyclohexanol and thereby produce a hydrogenation product containing cyclohexanone and cyclohexanol.

2. The process of claim 1, further comprising:
(b) obtaining a mixture comprising at least 90 wt % of cyclohexanone and cyclohexanol from the hydrogenation product.

3. The process of claim 1, wherein the weight ratio of cyclohexanone to phenol in the feed is from 0.1 to 10.0.

4. The process of claim 1, wherein the hydrogenation catalyst comprises at least one of palladium, platinum, ruthenium, rhodium, iridium, osmium, nickel, zinc, tin, cobalt and compounds and mixtures thereof.

5. The process of claim 4, wherein the hydrogenation catalyst comprises both platinum and palladium.

6. The process of claim 5, wherein the hydrogenation catalyst comprises platinum and palladium at a molar ratio from 0.1 to 10.0.

7. The process of claim 4, wherein the hydrogenation catalyst further comprises at least one of the following inorganic oxides: Al₂O₃, SiO₂, ZrO₂, MgO, CaO, Gd₂O₃, GeO₂, Y₂O₃, rare earth oxides, TiO₂, SnO₂, and combinations and mixtures thereof.

8. The process of claim 7, wherein the hydrogenation catalyst comprises at least one of SnO and SnO₂ and at least one of SiO₂ and Al₂O₃.

9. A process for producing a mixture of cyclohexanone and cyclohexanol, the process comprising:
(a) cleaving a cleavage feed comprising cyclohexylbenzene hydroperoxide to produce a cleavage effluent comprising phenol and cyclohexanone;
(b) obtaining a hydrogenation feed comprising phenol and/or cyclohexanone from at least a portion of the cleavage effluent; and
(c) contacting the hydrogenation feed with hydrogen in the presence of a hydrogenation catalyst in a hydrogenation reaction zone under hydrogenation conditions effective to convert at least part of the phenol and/or cyclohexanone in the hydrogenation feed into cyclohexanol and thereby produce a hydrogenation product containing cyclohexanone and cyclohexanol.

10. The process of claim 9, further comprising:
(d) obtaining a mixture comprising at least 90 wt % of cyclohexanone and cyclohexanol from the hydrogenation product.

11. The process of claim 9, wherein the obtaining step (b) comprises:
(b1) obtaining from the cleavage effluent a first fraction having a higher cyclohexanone concentration than the cleavage effluent and a second fraction having a lower cyclohexanone concentration than the cleavage effluent; and
(b2) forming the hydrogenation feed from at least a portion of the first fraction.

12. The process of claim 11, wherein the obtaining step (b1) comprises distillation.

13. The process of claim 9, wherein the hydrogenation catalyst in step (c) is selected from the group consisting of palladium, platinum, ruthenium, rhodium, iridium, osmium, nickel, zinc, tin, cobalt and compounds and mixtures thereof.

14. The process of claim 13, wherein the hydrogenation catalyst comprises both platinum and palladium.

15. The process of claim 14, wherein the hydrogenation catalyst comprise platinum and palladium at a molar ratio from 0.1 to 10.0.

16. The process of claim 9, wherein the hydrogenation catalyst further comprises at least one of the following inorganic oxides: Al₂O₃, SiO₂, ZrO₂, MgO, CaO, Gd₂O₃, GeO₂, Y₂O₃, rare earth oxides, TiO₂, SnO₂, and combinations and mixtures thereof.

17. The process of claim 16, wherein the hydrogenation catalyst comprises at least one of SnO and SnO₂ and at least one of SiO₂ and Al₂O₃.

18. The process of claim 9, wherein at least a portion of the cyclohexylbenzene hydroperoxide in step (a) is provided by:
(a1) contacting benzene and hydrogen with a hydroalkylation catalyst under hydroalkylation conditions to produce cyclohexylbenzene; and
(a2) oxidizing at least part of the cyclohexylbenzene from step (a1) to produce an oxidation effluent comprising cyclohexylbenzene hydroperoxide.

19. A hydrogenation catalyst comprising platinum, palladium, tin and an inorganic oxide material.

20. The hydrogenation catalyst of claim 19, wherein the molar ratio of platinum to palladium in the catalyst is from 0.1 to 10.0.

21. The hydrogenation catalyst of claim 19, wherein the inorganic oxide material comprises at least one of Al₂O₃, SiO₂, ZrO₂, MgO, CaO, Gd₂O₃, GeO₂, Y₂O₃, rare earth oxides, TiO₂, SnO, SnO₂, and combinations and mixtures thereof.

22. The hydrogenation catalyst of claim 21, wherein the inorganic oxide material comprises SiO₂ and/or Al₂O₃.

23. The hydrogenation catalyst of claim 22, wherein the inorganic oxide material comprises at least one of Al₂O₃ and SiO₂, and at least one of SnO and SnO₂.

24. The hydrogenation catalyst of claim 23, wherein the molar ratio of SnO to SnO₂ is from 0.001 to 1000.

25. The hydrogenation catalyst of claim 24, wherein the total concentration of platinum and palladium is from 0.001 wt % to 5.0 wt %, based on the total weight of the catalyst.
